(19) United States
(12) Patent Application Publication
(10) Pub. No.: US 2005/0217672 A1
(43) Pub. Date: Oct. 6, 2005

(54) Combined gas flow generator & control valve housing in a ventilator

(76) Inventors: Staffan Bengtsson, Goteborg (SE); Lars Ljungberg, Floda (SE); Mats Nilsson, Kungsbacka (SE); Mikael Tiedje, Hisings Backa (SE)

Correspondence Address: MOSER, PATTERSON & SHERIDAN L.L.P., 595 SHREWSBURY AVE, STE 100, FIRST FLOOR, SHREWSBURY, NJ, 07702, US

(21) Appl. No.: 11/099,208

(22) Filed: Apr. 5, 2005

Related U.S. Application Data
(60) Provisional application No. 60/573,230, filed on May 21, 2004.

(30) Foreign Application Priority Data
Apr. 5, 2004 (SE) 0400891-8

(51) Int. Cl. A62B 7/00; A61M 16/00

(52) U.S. Cl. 128/204.18

(57) ABSTRACT

A ventilator for supplying breathable gas to the airway of a patient with a respiratory disorder, comprising: a gas flow generator, such as an electric fan, for generating a flow of said breathable gas to the patient, said gas flow generator comprising a gas flow generator chamber provided with a gas inlet opening and a gas outlet opening; a control valve for controlling the flow and/or pressure of the gas distributed to the patient, said control valve comprising a valve body which is movably arranged within a valve chamber, wherein said gas flow generator chamber and said valve chamber are integrally formed in a combined gas flow generator & control valve housing; said valve chamber is located in immediate conjunction to the gas outlet opening of the gas flow generator chamber within said combined gas flow generator & valve housing; and said valve body comprise transitions portions for providing smooth pressure and/or flow transitions during transitions between closed and open positions of said control valve.

### CROSS REFERENCE TO RELATED APPLICATION

This application claims priority from Swedish Patent Application 0400891-8 filed on Apr. 5, 2004 and U.S. Provisional Patent Application Ser. No. 60/573,230, filed on May 21, 2004.

### FIELD OF THE INVENTION

The present invention relates to a ventilator for supplying breathable gas, normally air, at elevated pressure to a patient for treating breathing disorders such as for example Obstructive Sleep Apnea (OSA), Cheyne-Stokes respiration or emphysema. The ventilator may also be used in the treatment of cardiac disorders, such as Congestive Heart Failure (CHF). The invention is applicable to advanced intensive care ventilators for assisted ventilation or Continuous Positive Airway Pressure ventilators (CPAP). More particularly, the ventilator comprises a novel compact design feature, which substantially reduces the overall size of the ventilator, thus improving user comfort for the patients.

### BACKGROUND OF THE INVENTION

Ventilators for supplying breathable gas to the airway of a patient, are well known in the art per se. In the simplest form of CPAP therapy (not applicable in the present invention), air of a constant positive pressure is supplied to the airway of a patient, in order to treat Obstructive Sleep Apnea (OSA). The required pressure level varies for individual patients and their respective breathing disorders. CPAP therapy may be applied not only to the treatment of breathing disorders, but also to the treatment of Congestive Heart Failure (CHF).

A more advanced form of CPAP therapy is commonly referred to as Bi-Level CPAP, wherein air is applied to the airway of a patient alternatively at a higher pressure level during inspiration and a lower pressure level during expiration. The higher pressure level is referred to as IPAP (Inspiratory Positive Airway Pressure), whilst the lower pressure level is referred to as EPAP (Expiratory Positive Airway Pressure). In a Bi-level CPAP ventilator, EPAP and IPAP are thus synchronized with the patient's inspiratory cycle and expiratory cycle so that the patient will not be forced to overcome a high pressure from the ventilator during the expiration phase of his or her breathing. Consequently, Bi-Level CPAP ventilators generally provides improved breathing comfort for the patient compared to the simpler “single level” CPAP ventilator described initially. In order to detect the patient's transition from the inspiratory breathing phase to the expiratory breathing phase, a Bi-Level CPAP ventilator is provided with one or more sensors. Normally, a flow sensor is located somewhere along the air supply conduit to the patient. Additionally, a pressure sensor may for example be located in a patient interface means, such as a facial mask, or along the air supply conduit. The different pressure levels and/or flow levels are normally controlled by means of a control valve, which restricts and directs the airflow in various ways. As will be described in more detail below, modern ventilators often use a gas flow generator in the form of an electric fan unit, and the pressure and flow may thus be additionally or exclusively controlled by varying the rotary speed of the fan.

Another, yet more advanced type of CPAP ventilator is generally referred to as an AutoCPAP ventilator. Other terms for this type of ventilator include: Auto Adaptive CPAP (AACPAP), Auto Titration CPAP or Self-titrating individual AutoCPAP. In this description, these terms will commonly be referred to as an AutoCPAP ventilator for the sake of clarity. Here, IPAP and EPAP as well as other relevant parameters are automatically changed with respect to specific detected breathing patterns significative of different breathing disorders or phases thereof. This is an “intelligent” form of CPAP treatment, in which a certain condition may even be foreseen by the ventilator before the condition is felt by the patient, and wherein a suitable combination of IPAP and EPAP as well as other relevant parameters are applied in order to treat or alleviate the symptoms of the patient. For this purpose, it is known to provide a ventilator with an integral learning artificial neural network (ANN) to gather large amounts of relevant breathing data from a vast population of patients with breathing disorders worldwide. The ANN is able to detect and identify breathing patterns that are symptomatic of a certain condition or disorder and to then automatically adapt the ventilator parameter settings for effecting a relevant treatment pattern at an early stage. Apart from added control hardware, software and more sensors, the basic hardware design of an AutoCPAP ventilator may be substantially identical a Bi-Level CPAP ventilator.

A trend in modern ventilator technology is directed toward ever more compact and lightweight CPAP ventilators, that are unobtrusive at the bedside, offer increased mobility for patients and generally have a less “hospital-like” design, in order to improve user comfort.

A ventilator of the above mentioned type includes a gas flow generator for creating a gas flow to the patient. A patient interface means, in the form of a facial mask or a tracheal tube is provided for introducing the breathable gas into the airway of the patient.

In older ventilators, the gas flow generator often consisted of an air bellows unit, which was sufficiently quiet, but had to be rather large in order to effectively produce the required airflow. Thus, in modern, more compact ventilators, a compact but effective electric fan unit has replaced the air bellows often found in older systems.

In the more the advanced CPAP ventilators, such as the Bi-level CPAP or AACPAP mentioned above, a control valve is provided for controlling the flow and/or pressure of the gas from the gas flow generator. The simplest form of CPAP ventilator lacks this feature, and is thus not covered by the present invention. The control valve comprises a valve body, which is movably arranged within a valve chamber.

However, even in the more modern conventional ventilators, the control valve is traditionally designed and manufactured as a separate assembly within the ventilator and is connected to the gas flow generator by means of an interconnecting pipe or hose conduit section of various lengths depending on the layout of a specific ventilator. Hence, partly for this reason, conventional ventilators tend to be unnecessarily bulky.

### OBJECT OF THE INVENTION

It is the object of the present invention to provide a more compact ventilator compared to currently available ventilators on the market, as well as to reduce the overall manufacturing cost of the ventilator.

### SUMMARY OF THE INVENTION

The above mentioned object is achieved by the invention providing a ventilator for supplying breathable gas to the airway of a patient with a respiratory disorder, comprising:
a gas flow generator, such as an electric fan, for generating a flow of said breathable gas to the patient, said gas flow generator comprising a gas flow generator chamber provided with a gas inlet opening and a gas outlet opening;
a control valve for controlling the flow and/or pressure of the gas distributed to the patient, said control valve comprising a valve body which is movably arranged within a valve chamber wherein, said gas flow generator chamber and said valve chamber are integrally formed in a combined gas flow generator & control valve housing; said valve chamber is located in immediate conjunction to the gas outlet opening of the gas flow generator chamber within said combined gas flow generator & valve housing and said valve body comprise transitions portions for providing smooth pressure and/or flow transitions during transitions between closed and open positions of the valve body.

In an advantageous embodiment of the invention, the gas outlet opening of the gas flow generator chamber also defines an inlet opening to said valve chamber.

In one embodiment, the gas flow generator comprises a fan rotor wheel driven by an electric motor, said fan rotor wheel being arranged in said gas flow generator chamber. This embodiment is especially characterized in that the gas outlet opening of the gas flow generator chamber and the inlet opening of said valve chamber are formed in a peripheral outer wall of said gas flow generator chamber.

In an advantageous embodiment, a bypass conduit is also integrally formed in said combined gas flow generator & control valve housing. The bypass conduit extends from a bypass opening in the valve chamber to the gas inlet opening in the gas flow generator chamber. The bypass conduit extends—at least along a section of its length—along said outer peripheral wall of said gas flow generator chamber. Hereby, said outer peripheral wall of the gas flow generator chamber also defines an inner peripheral wall for the bypass conduit.

In a preferred embodiment, the combined gas flow generator & control valve housing is structurally divided—in a plane perpendicular to a rotational axis of the fan rotor wheel—into a first shell and a second shell, a section of said valve chamber being defined in each of said shells.

In the preferred embodiment, the valve body is rotatably arranged about a rotational axis parallel to said rotational axis of the fan rotor wheel.

Advantageously, an electric stepper motor is attached to the combined gas flow generator & control valve housing. The electric stepper motor has a stepper motor shaft coupled to the valve body in said valve chamber. Furthermore, the valve body is provided with a through hole, said through hole having a cross-sectional shape such that the valve body is rotationally fixed relative to the stepper motor shaft, whilst being freely slidably arranged in an axial direction of said stepper motor shaft for easy insertion or removal of the valve body in the valve chamber.

In a well functioning embodiment of the invention, the control valve chamber has a generally circular-cylindrical wall extending in the direction of said rotational axis of the valve body, said valve body having a cylindrical valve surface adapted for abutting contact with said circular-cylindrical wall.

Preferably, the combined gas flow generator & control valve housing is made in plastic by means of injection molding.

Further features and advantages of the invention will be described in the detailed description of embodiments below.

### BRIEF DESCRIPTION OF THE DRAWINGS

The invention will now be described in greater detail by way of example only and with reference to the attached drawings, in which

FIG. 1 shows a schematic view of a combined gas flow generator & control valve housing of a ventilator according to the invention;

FIG. 2 shows a perspective view of a more detailed combined gas flow generator & control valve housing according to the invention, shown with one shell removed in order to expose the internals of the housing, and

FIG. 3 shows an elevation view of the housing in FIG. 2 from the opposite side, illustrating the gas inlet opening to the gas flow generator chamber and the stepper motor attached to the housing.

FIG. 4 shows an elevation view of a valve according to the present invention.

### DESCRIPTION OF EXEMPLIFYING EMBODIMENTS

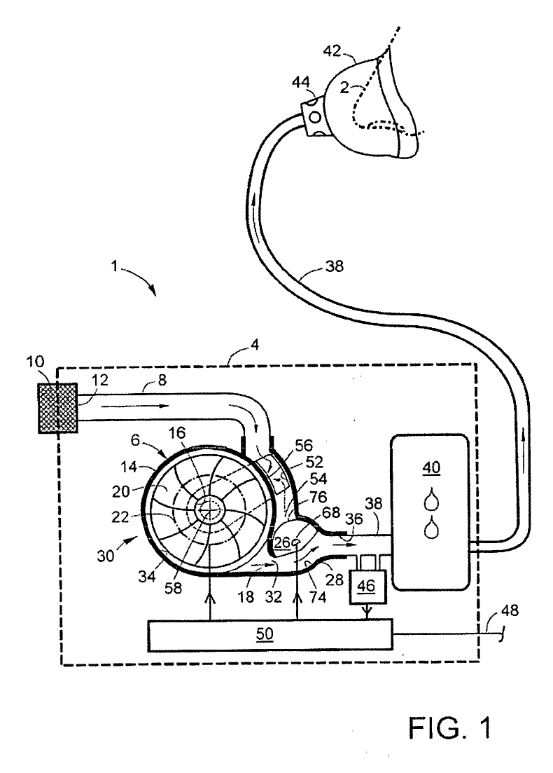

In FIG. 1, reference numeral 1 denotes a ventilator for supplying breathable gas—normally air—into the airway of a patient for treating breathing disorders such as for example Obstructive Sleep Apnea (OSA), Cheyne-Stokes respiration or emphysema. In the figure, a schematically drawn nose 2 of a patient is shown with dash-dotted lines. It should be noted that the schematic FIG. 1 is drawn in a highly simplified way in order to clearly illustrate the basic features of the invention. Thus, a production version of a ventilator according to the invention may look significantly different than in the shown illustrations, although the basic features are still present.

As mentioned in the background above, the ventilator is either of the initially described Bi-Level CPAP type or the AutoCPAP-type.

The ventilator 1 has an external housing 4, schematically illustrated with dashed lines in FIG. 1. A gas flow generator 6 is located within the external housing 4. In the preferred example, the gas flow generator 6 is an electric fan, adapted for generating a flow of breathable gas to the patient. The gas flow generator 6 draws in air (or any other breathable gas) via a gas inlet conduit 8. A particle filter 10 is provided at an external opening 12 of the gas inlet conduit 8 in order to stop undesired particular matter from entering the ventilator 1.

More particularly, the gas flow generator 6 comprises a generally circular gas flow generator chamber 14 provided with a gas inlet opening 16 and a gas outlet opening 18, respectively. As shown in FIG. 1, the gas flow generator 6 further comprises a fan rotor wheel 20 arranged within the gas flow generator chamber 14. The fan rotor wheel 20 is driven by an electric motor 22, which is schematically drawn with dash-dotted lines behind the fan rotor wheel 20. The electric motor 22 is preferably of a known compact type, wherein a stator (not shown) is fixedly attached to the combined gas flow generator & valve housing 30, and a rotor (not shown) is fixedly attached to said fan rotor wheel 20, the latter of course being rotatably journalled in the housing 30.

The ventilator 1 further comprises a control valve 24 for controlling the flow and/or pressure of the gas distributed to the patient. The control valve 24, in turn, comprises a valve body 26, which is movably arranged within a valve chamber 28.

As is clearly shown in FIG. 1, the gas flow generator chamber 14 and the valve chamber 28 are integrally formed in a combined gas flow generator & control valve housing 30, as drawn with bold black lines in the schematical FIG. 1. Moreover, the valve chamber 28 is located in immediate conjunction to the gas outlet opening 18 of the gas flow generator chamber 14 within said combined gas flow generator & valve housing 30. By the term “immediate conjunction” is here meant no intermediate conduit extends between the gas flow generator chamber 14 and the valve chamber 28. Thus, in the shown embodiment, the gas outlet opening 18 of the gas flow generator chamber 14 also defines an inlet opening 32 to said valve chamber 28.

In the embodiment shown in FIG. 1, the gas outlet opening 18 of said gas flow generator chamber 14 and said inlet opening 32 of said valve chamber 28 are formed in a peripheral outer wall 34 of said gas flow generator chamber 14.

As seen in the right end of the combined gas flow generator & control valve housing 30 in FIG. 1, the valve chamber 28 is also provided with an outlet opening 36. The outlet opening 36 is connected to an outlet conduit 38, which via an air humidifier 40 is connected to a patient interface means 42. The air humidifier 40 may be of a type well known per se and will thus not be described further here. The patient interface means 42 is adapted for introducing the breathable gas into the airway of said patient, and here includes a facial mask adapted for non-invasive attachment over the nose 2 of a patient. Exhaust openings 44, or “leakage holes” for venting exhaled air from the patient are provided on the patient interface means 42. The exhaust openings 44 may also include a valve (not shown). Alternatively, the patient interface means 42 instead includes a tracheal tube (not shown) for invasive insertion in the trachea of a patient. The external extension of the outlet conduit 38 is preferably a flexible hose.

In the shown example, a flow sensor 46 is located along the outlet conduit 38. The flow sensor 46, along with other optional sensors (Not shown, but as indicated as a symbolic input line 48) provides input for a control unit 50. The control unit 50 then controls either the speed of the electric motor 22, and thereby the fan rotor wheel 20, or the position of the valve body 26 within the valve chamber 28, or both, in order to provide an appropriate gas flow or pressure to the patient, depending—for example—on if he or she is in an inspiratory phase or an expiratory phase of breathing. Many ways and modes of controlling a Bi-Level CPAP or an AutoCPAP ventilator are known in the art, and will thus not be further described herein. In FIG. 1, the valve body 26 is in a fully open position, wherein full gas flow provided by the gas flow generator 6 is distributed to the patient, as indicated by the arrows.

In some situations, requiring a lesser gas flow to the patient, some air is passed by the control valve 24 and back into the gas flow generator via a bypass conduit 52, in a manner well known per se. However, in the embodiment shown in FIG. 1, the bypass conduit 52 is integrally formed in the combined gas flow generator & control valve housing 30. The bypass conduit 52 extends from a bypass opening 54 in the valve chamber 28 to the gas inlet opening 16 in the gas flow generator chamber 14.

The bypass conduit 52 extends—at least along a section of its length—along the peripheral outer wall 34 of said gas flow generator chamber 14, said peripheral outer wall here also defining a peripheral inner wall 56 for the bypass conduit 52.

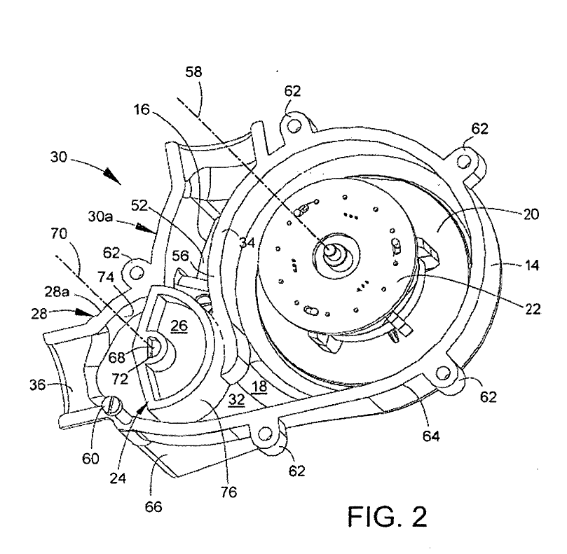

In a preferred embodiment as shown in FIG. 2, the combined gas flow generator & control valve housing 30 is structurally divided—in a plane perpendicular to a rotational axis 58 of the fan rotor wheel 20—into a first shell 30a and a second shell 30b (not shown), i.e. the plane of the paper sheet in FIG. 1. FIG. 2 shows the housing 30 with one shell, 30b, removed in order to expose the internals of the housing 30. Hereby, a section 28a, 28b (not shown) of the valve chamber 28 is defined in each of the shells 28a and 28b (not shown). Preferably the two shells 30a, 30b of the combined gas flow generator & control valve housing 30 are made in plastic by means of injection molding. Alternatively, however, the shells 30a, 30b may be made in metal, such as zinc or bronze. In the shown embodiment, the shells 30a and 30b are joined together by means of multiple mounting screws 60 (only one of which is shown) extending through a corresponding number of screw lugs 62 located along the outline periphery 64 of each shell 30a, 30b. A skilled man in the art will of course realize that the shells may alternatively be joined together in other ways, such as for example by means of snap fasteners (not shown).

As is further shown in FIG. 2, an electric stepper motor 66 is attached to the combined gas flow generator & control valve housing 30, said electric stepper motor having a stepper motor shaft 68 coupled to the valve body 26 in the valve chamber 28. The valve body 28 is thus rotatably arranged about a rotational axis 70—which coincides with the stepper motor shaft 68 and extends parallelly with previously mentioned rotational axis 58 of the fan rotor wheel 20. In FIG. 2, said axis 70 and the rotational axis 58 are illustrated with dash-dotted lines for clarity. The electric stepper motor 66 may alternatively be replaced by another type of motor or turning rotating device adopted to rotate the valve body 26.

The valve body 26 is provided with a through hole 72 for the stepper motor shaft 68. The through hole 72 has a cross-sectional shape such that the valve body 26 is rotationally fixed relative to the stepper motor shaft 68, whilst being freely slidably arranged in an axial direction of said stepper motor shaft 68 for easy insertion or removal of the valve body 26 in the valve chamber 28. In the shown example, the cross-sectional shape is semi-circular, but other equally suitable shapes may alternatively be used for the same purpose, such as triangular, rectangular, pentagonal, hexagonal or other polygonal shapes. As illustrated by the semi-circular shape, the cross-sectional shape may also be partially rounded.

In FIGS. 1 and 2, it is shown that the control valve chamber 28 has a generally circular-cylindrical wall 74 extending in the direction of said rotational axis 70 of the valve body 26. As is further shown, the valve body 26 has a cylindrical valve surface 76 adapted for abutting contact with said circular-cylindrical wall 74.

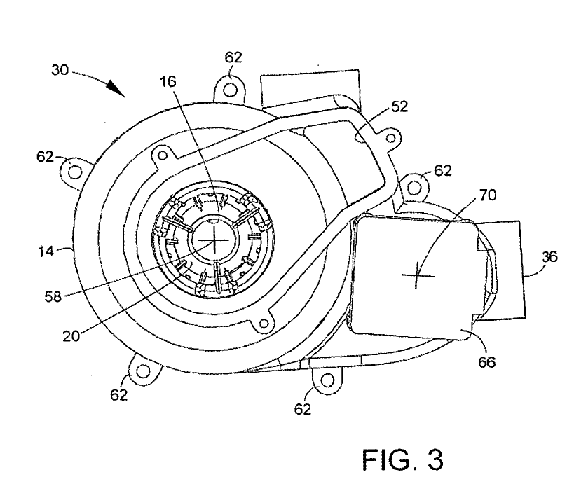

FIG. 3 shows an elevation view of the combined gas flow generator & control valve housing 30 in FIG. 2 from the opposite side, illustrating the gas inlet opening 16 to the gas flow generator chamber 14 and the stepper motor 66 attached to the housing 30.

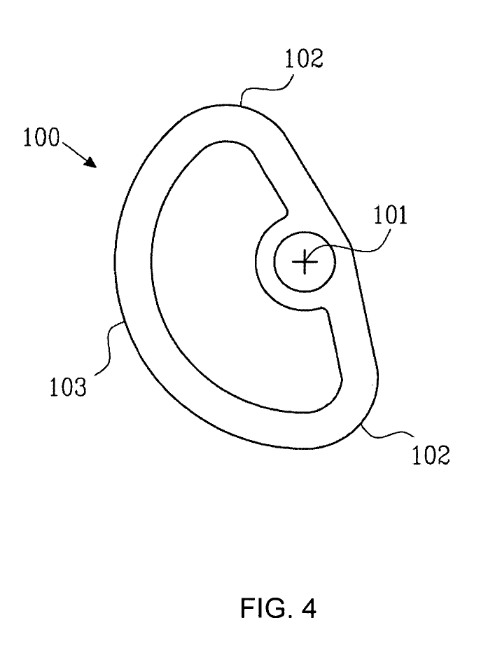

FIG. 4 shows an elevation view of the control valve body 26 (marked with 100 in this figure). The valve body is rotatable around an axis 101 and in this figure the cylindrical valve surface of the valve body 101 is marked with 103 (76 previously). The valve body 100 has two transitional portions 102 in order to provide smooth pressure or flow, or pressure and flow transitions between open and closed positions of the valve body 100. When the valve body 100 is moved to a closed or open position, the transition portions 102 provide a slow transition between the open and closed position (and vice versa) of the control valve due to the radius of the transition portions 102. This will have the effect of a more comfortable feeling for the user of the ventilator.

It is to be understood that the invention is by no means limited to the embodiments described above, and may be varied freely within the scope of the appended claims.

### LIST OF REFERENCE NUMERALS AND SIGNS

1. Ventilator
2. Schematic illustration of a patients nose
4. External housing
6. Gas flow generator
8. Gas inlet conduit
10. Particle filter
12. External opening of the gas inlet conduit
14. Gas flow generator chamber
16. Gas inlet opening in gas flow generator chamber
18. Gas outlet opening in gas flow generator chamber
20. Fan rotor wheel
22. Electric motor
24. Control valve
26. Valve body
28. Valve chamber
28a. Section of valve chamber
28b. Section of valve chamber (not shown)
30. Combined gas flow generator & control valve housing
30a. First shell
30b. Second shell (not shown)
32. Inlet opening to valve chamber
34. Peripheral outer wall of gas flow generator chamber
36. Outlet opening of valve chamber
38. Outlet conduit
40. Air Humidifier
42. Patient interface means (facial mask)
44. Exhaust openings
46. Flow sensor
48. Other optional sensors
50. Control unit
52. Bypass conduit
54. Bypass opening
56. Peripheral inner wall of bypass conduit
58. Rotational axis 58 of fan rotor wheel
60. Mounting screws
62. Screw lugs
64. Outline periphery of the shells
66. Electric stepper motor
68. Stepper motor shaft
70. Rotational axis of valve body
72. Trough hole in valve body for stepper motor shaft
74. Circular-cylindrical wall of control valve chamber
76. Cylindrical valve surface of valve body
100. Valve body
101. Axis
102. Transition portions
103. Cylindrical valve surface of valve body

1. Ventilator for supplying breathable gas to the airway of a patient with a respiratory disorder, comprising:
a gas flow generator, such as an electric fan, for generating a flow of said breathable gas to the patient, said gas flow generator comprising a gas flow generator chamber provided with a gas inlet opening and a gas outlet opening;
a control valve for controlling the flow and/or pressure of the gas distributed to the patient, said control valve comprising a valve body which is movably arranged within a valve chamber, wherein said gas flow generator chamber and said valve chamber are integrally formed in a combined gas flow generator & control valve housing; said valve chamber is located in immediate conjunction to the gas outlet opening of the gas flow generator chamber within said combined gas flow generator & valve housing, and said valve body comprise transitions portions for providing smooth pressure and/or flow transitions during transitions between closed and open positions of said control valve.

2. Ventilator according to claim 1, wherein said gas outlet opening of the gas flow generator chamber also defines an inlet opening to said valve chamber.

3. Ventilator according to claim 2, wherein said gas flow generator comprises a fan rotor wheel driven by an electric motor, said fan rotor wheel being arranged in said gas flow generator chamber, wherein said gas outlet opening of said gas flow generator chamber and said inlet opening of said valve chamber are formed in a peripheral outer wall of said gas flow generator chamber.

4. Ventilator according to claim 3, wherein a bypass conduit is integrally formed in said combined gas flow generator & control valve housing, said bypass conduit extending from a bypass opening in the valve chamber to the gas inlet opening in the gas flow generator chamber.

5. Ventilator according to claims 4, wherein said bypass conduit—at least along a section of its length—extends along said peripheral outer wall of said gas flow generator chamber, said peripheral outer wall defining a peripheral inner wall for the bypass conduit.

6. Ventilator according to any of claims 5, wherein the combined gas flow generator & control valve housing is structurally divided—in a plane perpendicular to a rotational axis of the fan rotor wheel—into a first shell and a second shell, a section of said valve chamber being defined in each of said shells.

7. Ventilator according to claim 6, wherein the valve body is rotatably arranged about a rotational axis parallel to said rotational axis of the fan rotor wheel.

8. Ventilator according to claim 1, wherein an electric stepper motor is attached to the combined gas flow generator & control valve housing, said electric stepper motor having a stepper motor shaft coupled to the valve body in said valve chamber.

9. Ventilator according to claim 8, wherein the valve body is provided with a through hole, said through hole having a cross-sectional shape such that the valve body is rotationally fixed relative to the stepper motor shaft, whilst being freely slidably arranged in an axial direction of said stepper motor shaft for easy insertion or removal of the valve body in the valve chamber.

10. Ventilator according to claim 1, wherein the control valve chamber has a generally circular-cylindrical wall extending in the direction of said rotational axis of the valve body, said valve body having a cylindrical valve surface adapted for abutting contact with said circular-cylindrical wall.

11. Ventilator according to claim 1, wherein the combined gas flow generator & control valve housing is made in plastic by means of injection molding.
